(19) United States
(12) Patent Application Publication
(10) Pub. No.: US 2021/0011887 A1
(43) Pub. Date: Jan. 14, 2021

(54) ACTIVITY QUERY RESPONSE SYSTEM

(71) Applicant: QUALCOMM Incorporated, San Diego, CA (US)

(72) Inventors: Erik VISSER, San Diego, CA (US); Rehana MAHFUZ, West Lafayette, IN (US); Ravi CHOUDHARY, San Diego, CA (US); Lae-Hoon KIM, San Diego, CA (US); Sunkuk MOON, San Diego, CA (US); Yinyi GUO, San Diego, CA (US); Fatemeh SAKI, San Diego, CA (US)

(21) Appl. No.: 16/586,821

(22) Filed: Sep. 27, 2019

Related U.S. Application Data
(60) Provisional application No. 62/873,768, filed on Jul. 12, 2019.

(51) Int. Cl. G06F 16/18; G06F 17/28; G06N 3/04; G06F 16/61; A61B 5/11

(52) U.S. Cl. CPC G06F 16/1805; G06F 17/28; A61B 5/1118; G06F 16/61; G06N 3/04

(57) ABSTRACT

A device for activity tracking includes a memory and one or more processors. The memory is configured to store an activity log. The one or more processors are configured to update the activity log based on activity data. The activity data is received from a second device. The one or more processors are also configured to, responsive to receiving a natural language query, generate a query response based on the activity log.

### I. CROSS-REFERENCE TO RELATED APPLICATIONS

The present application claims priority from U.S. Provisional Patent Application No. 62/873,768, filed Jul. 12, 2019, entitled “ACTIVITY QUERY RESPONSE SYSTEM,” which is incorporated by reference in its entirety.

### II. FIELD

The present disclosure is generally related to generating activity query responses.

### III. DESCRIPTION OF RELATED ART

Advances in technology have resulted in smaller and more powerful computing devices. For example, there currently exist a variety of portable personal computing devices, including wireless telephones such as mobile and smart phones, tablets and laptop computers that are small, lightweight, and easily carried by users. These devices can communicate voice and data packets over wireless networks. Further, many such devices incorporate additional functionality such as a digital still camera, a digital video camera, a digital recorder, and an audio file player. Also, such devices can process executable instructions, including software applications, such as a web browser application, that can be used to access the Internet. As such, these devices can include significant computing capabilities.

Devices with sensors are becoming increasingly ubiquitous. The data that can be generated by such devices can be used to determine useful information in various applications. For example, sensor data from a security system can indicate suspicious activity. As another example, sensor data from home appliances can indicate to a caregiver whether a particular person has taken food out of a refrigerator. Analyzing the large amount of data generated by such sensors uses relatively large memory resources and processing resources that may not be available at a local device. However, sending the sensor data to an external network for processing raises privacy concerns.

### IV. SUMMARY

In a particular aspect, a device for activity tracking includes a memory and one or more processors. The memory is configured to store an activity log. The one or more processors are configured to update the activity log based on activity data. The activity data is received from a second device. The one or more processors are also configured to, responsive to receiving a natural language query, generate a query response based on the activity log.

In another particular aspect, a method of activity tracking includes receiving activity data at a first device from a second device. The method also includes updating, at the first device, an activity log based on the activity data. The method further includes, responsive to receiving a natural language query, generating a query response based on the activity log.

In another particular aspect, a computer-readable storage device includes instructions that, when executed by one or more processors, cause the one or more processors to update an activity log based on activity data received from a device. The instructions, when executed by the one or more processors, cause the one or more processors to, responsive to receiving a natural language query, generate a query response based on the activity log.

In another particular aspect, an apparatus for activity tracking includes means for storing an activity log. The apparatus also includes means for updating the activity log based on activity data. The activity data is received from a second device. The apparatus further includes means for generating a query response based on the activity log. The query response is generated responsive to receiving a natural language query.

Other aspects, advantages, and features of the present disclosure will become apparent after review of the entire application, including the following sections: Brief Description of the Drawings, Detailed Description, and the Claims.

### V. BRIEF DESCRIPTION OF THE DRAWINGS

FIG. 1 is a block diagram of a particular illustrative aspect of an activity query response system;

FIG. 2A is a diagram of an example of activity detection that may be performed by the system of FIG. 1;

FIG. 2B is a diagram of an example of activity detection that may be performed by the system of FIG. 1;

FIG. 3 is a diagram of an example of an activity log, queries, and answers that may be generated by the system of FIG. 1;

FIG. 4 is a diagram of another example of an activity log, a query, and a query response that may be generated by the system of FIG. 1;

FIG. 5 is a diagram of another example of an activity log, a query, and a query response that may be generated by the system of FIG. 1;

FIG. 6 is a flow chart illustrating a particular method of activity query response generation; and

FIG. 7 is a block diagram of a particular illustrative example of a device that is operable to generate activity query responses.

### VI. DETAILED DESCRIPTION

System and methods for generating activity query responses are disclosed. An activity tracker generates a text-based activity log that indicates activities detected by sensors of local devices. A query response system receives natural language queries and generates query responses by using artificial intelligence techniques to analyze the activity log. In some examples, the activity tracker and the query response system are integrated into a device that is isolated from external networks. A text-based activity log combined with artificial intelligence techniques (e.g., machine learning) of the query response system enables generating query responses for natural language queries using relatively few processing and memory resources. Using fewer processing and memory resources enables the activity tracking and query response generation to be performed locally to increase privacy, as compared to cloud-based processing of activity data.

Particular aspects of the present disclosure are described below with reference to the drawings. In the description, common features are designated by common reference numbers. As used herein, various terminology is used for the purpose of describing particular implementations only and is not intended to be limiting of implementations. For example, the singular forms “a,” “an,” and “the” are intended to include the plural forms as well, unless the context clearly indicates otherwise. It may be further understood that the terms “comprise,” “comprises,” and “comprising” may be used interchangeably with “include,” “includes,” or “including.” Additionally, it will be understood that the term “wherein” may be used interchangeably with “where.” As used herein, “exemplary” may indicate an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to one or more of a particular element, and the term “plurality” refers to multiple (e.g., two or more) of a particular element.

As used herein, “coupled” may include “communicatively coupled,” “electrically coupled,” or “physically coupled,” and may also (or alternatively) include any combinations thereof. Two devices (or components) may be coupled (e.g., communicatively coupled, electrically coupled, or physically coupled) directly or indirectly via one or more other devices, components, wires, buses, networks (e.g., a wired network, a wireless network, or a combination thereof), etc. Two devices (or components) that are electrically coupled may be included in the same device or in different devices and may be connected via electronics, one or more connectors, or inductive coupling, as illustrative, non-limiting examples. In some implementations, two devices (or components) that are communicatively coupled, such as in electrical communication, may send and receive electrical signals (digital signals or analog signals) directly or indirectly, such as via one or more wires, buses, networks, etc. As used herein, “directly coupled” may include two devices that are coupled (e.g., communicatively coupled, electrically coupled, or physically coupled) without intervening components.

In the present disclosure, terms such as “determining”, “calculating”, “estimating”, “shifting”, “adjusting”, etc. may be used to describe how one or more operations are performed. It should be noted that such terms are not to be construed as limiting and other techniques may be utilized to perform similar operations. Additionally, as referred to herein, “generating”, “calculating”, “estimating”, “using”, “selecting”, “accessing”, and “determining” may be used interchangeably. For example, “generating”, “calculating”, “estimating”, or “determining” a parameter (or a signal) may refer to actively generating, estimating, calculating, or determining the parameter (or the signal) or may refer to using, selecting, or accessing the parameter (or signal) that is already generated, such as by another component or device.

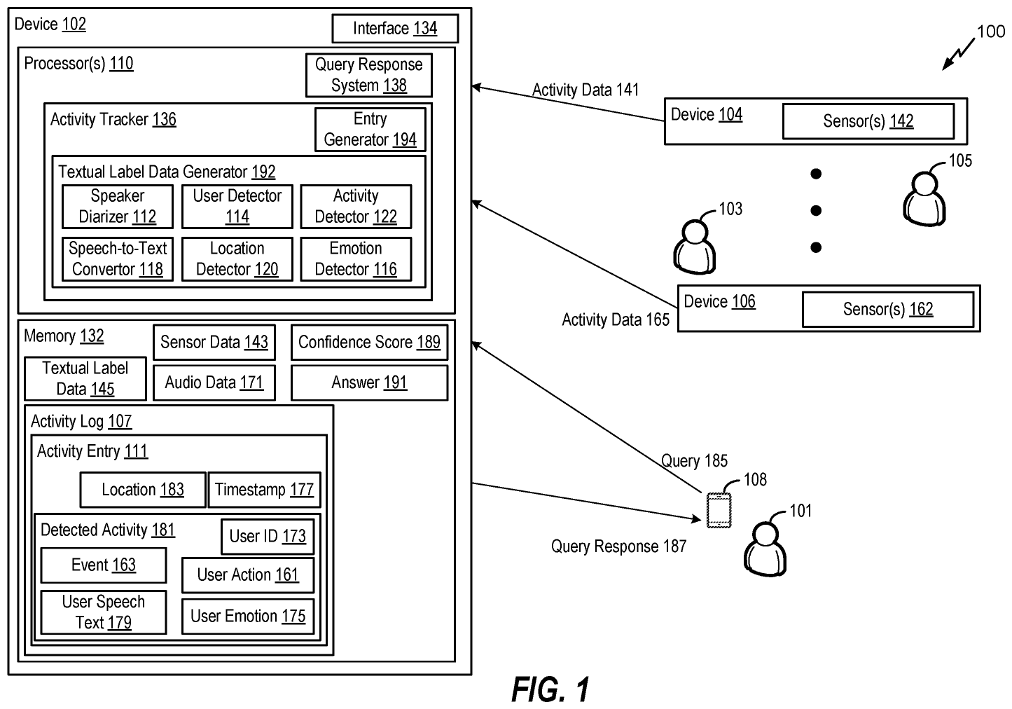

Referring to FIG. 1, a particular illustrative aspect of a system is disclosed and generally designated 100. The system 100 (e.g., an activity query response system) includes a device 102 communicatively coupled, via an interface 134, to a device 104, a device 106, a device 108, one or more additional devices, or a combination thereof. In a particular aspect, the device 104, the device 106, the device 108, or a combination thereof, can enter or leave a communication range of the device 102 at various times.

In a particular example, the device 102 is communicatively coupled, via the interface 134, to one or more local devices. In a particular aspect, a “local device” refers to a device that is within a threshold distance (e.g., 70 feet) of the device 102. In a particular aspect, a “local device” refers to a device that is coupled to the device 102 via at least one of a local area network or a peer-to-peer network. In a particular example, the local devices include at least one of the device 104, the device 106, or the device 108. In a particular aspect, the interface 134 is configured to enable local wireless networking with one or more local devices and to isolate the device 102 from external networks (e.g., to prevent interaction or data transfer from the device 102 to a public network or cloud-based computing system in accordance with a privacy policy).

In a particular aspect, one or more of the device 102, the device 104, the device 106, or the device 108 includes at least one of a portable electronic device, a home appliance, factory equipment, a security device, a vehicle, a car, an internet-of-things (IoT) device, a television, an entertainment device, a navigation device, a fitness tracker, a mobile device, a health monitor, a communication device, a computer, a virtual reality device, an augmented reality device, or a device controller. The device 104 and the device 106 include one or more sensors 142 and one or more sensors 162, respectively. A sensor includes at least one of an audio sensor (e.g., a microphone), an image sensor (e.g., a camera), a motion sensor, an open-close sensor, a weight scale, a remote control, or an input interface, as illustrative non-limiting examples.

The device 104 and the device 106 are configured to generate activity data 141 and activity data 165, respectively. In a particular example, the device 104 is configured to receive sensor data 143 from the sensor(s) 142. In a particular aspect, the device 104 is configured to generate textual label data 145 based on the sensor data 143 and to send the textual label data 145 as the activity data 141 to the device 102. In another aspect, the device 104 is configured to send the sensor data 143 as the activity data 141 to the device 102, and the device 102 is configured to generate the textual label data 145 based on the sensor data 143. The textual label data 145 indicates a detected activity 181, a location 183 of the detected activity 181, or both, as described herein. In a particular aspect, the activity data 141 indicates a device identifier of the device 104, a location 183 of the device 104, a timestamp 177, or a combination thereof. In a particular aspect, the timestamp 177 indicates a creation time of the sensor data 143, indicates a time at which the sensor data 143 is received by the device 104, a time at which the textual label data 145 is generated, a time at which the activity data 141 is transmitted to the device 102, or a combination thereof.

The device 102 includes a memory 132 configured to store an activity log 107. The device 102 includes one or more processors 110 coupled to the memory 132, the interface 134, or both. The processors 110 include an activity tracker 136, a query response system 138, or both. The activity tracker 136 is configured to generate (or update) the activity log 107 based on the activity data 141, as described herein. The query response system 138 is configured to generate, based on the activity log 107, a query response 187 that is responsive to a query 185, as described herein.

The activity tracker 136 includes textual label data generator 192, an entry generator 194, or both. The textual label data generator 192 is configured to generate textual label data based on activity data, as described herein. For example, the textual label data generator 192 is configured to generate the textual label data 145 based on the activity data 141, second textual label data based on the activity data 165, additional textual label data based on activity data received from one or more additional devices, or a combination thereof. The entry generator 194 is configured to generate an activity entry 111 based on textual label data. For example, the entry generator 194 is configured to generate the activity entry 111 based on the textual label data 145, the second textual label data, the additional textual label data, or a combination thereof. The activity tracker 136 is configured to generate (or update) the activity log 107 by adding the activity entry 111 to the activity log 107.

The textual label data generator 192 includes a speaker diarizer 112, a user detector 114, an emotion detector 116, a speech-to-text convertor 118, a location detector 120, an activity detector 122, or a combination thereof. The speaker diarizer 112 is configured to identify audio data corresponding to a single speaker. For example, the speaker diarizer 112 is configured to analyze the sensor data 143 (e.g., speech data) and identify portions of the sensor data 143 as audio data 171 corresponding to an individual speaker. The user detector 114 is configured to detect user 105 (associated with a user identifier (ID) 173) based on performing facial recognition, voice recognition, thumbprint analysis, retinal analysis, user input, one or more other techniques, or a combination thereof, on the sensor data 143. In a particular aspect, the user detector 114 is configured to detect the user 105 by performing voice recognition on the audio data 171. The speech-to-text convertor 118 is configured to convert the sensor data 143 (e.g., the audio data 171) including speech of the user 105 to user speech text 179 (e.g., a textual representation) indicating the speech of the user.

The location detector 120 is configured to detect that the location 183 is associated with the sensor data 143. In a particular example, the location detector 120 detects that the location 183 is associated with the sensor data 143 in response to determining that sensor data 143 indicates the location 183, the activity data 141 indicates the location 183, that the device 104 is associated with (assigned to) the location 183, or a combination thereof. The activity detector 122 is configured to detect, based on the sensor data 143, an event 163, a user action 161, or both. For example, detecting the user action 161 includes determining that the sensor data 143 indicates presence of the user 105. In another example, detecting the user action 161 includes detecting one or more actions performed by the user 105. Illustrative non-limiting examples of the user action 161 include opening a door, changing a device setting, performing a gesture, playing a musical instrument, watching television, reading a book, exercising, eating, or a combination thereof In a particular example, detecting the event 163 includes detecting events that are not actions performed by a user. Illustrative non-limiting examples of the event 163 include a sound of breaking glass, activation of an alarm, a status update of the sensor(s) 142, or a combination thereof.

The emotion detector 116 is configured to detect a user emotion 175 based on the sensor data 143. For example, the emotion detector 116 is configured to detect the user emotion 175 by performing a voice analysis (e.g., voice quality, such as pitch or loudness), speech analysis (e.g., particular words used in speech), facial expression analysis, gesture analysis, or a combination thereof. In a particular aspect, the emotion detector 116 is configured to detect the user emotion 175 by performing a voice analysis of the audio data 171, performing speech analysis of the user speech text 179, performing action analysis (e.g., gesture analysis) of the user action 161, facial expression analysis of the sensor data 143 (e.g., image sensor data), or a combination thereof.

During operation, the sensor(s) 142 of the device 104 generate the sensor data 143. In a particular example, the user 105 is speaking within a coverage area of the sensor(s) 142 and the sensor(s) 142 generate the sensor data 143 including the audio data 171 corresponding to speech of the user 105. In a particular aspect, the sensor data 143 includes speech of multiple speakers, e.g., the user 105 and a user 103. It should be understood that the sensor data 143 including the audio data 171 is provided as an illustrative example. In other examples, the sensor data 143 includes image sensor data, temperature sensor data, non-speech audio data, or other types of sensor data.

In a particular aspect, the device 104 sends the sensor data 143 as the activity data 141 to the device 102. In another aspect, the device 104 generates textual label data 145 based on the sensor data 143. For example, in some implementations, the device 104 is configured to perform one or more operations described with reference to the textual label data generator 192. The device 104 sends the textual label data 145 as the activity data 141 to the device 102. In a particular aspect, the activity data 141 indicates the location 183, the timestamp 177, or both. In a particular aspect, the device 104 sends the activity data 141 to the device 102 in response to detecting an event, such as expiration of a timer, generation of the sensor data 143 by the sensor(s) 142, receipt of a request from the device 102, or a combination thereof.

The device 102 receives the activity data 141 from the device 104. In a particular aspect, the device 102 receives activity data from multiple devices. For example, the device 102 receives activity data 165 from the device 106 concurrently with receiving the activity data 141 from the device 104.

The activity tracker 136, in response to determining that the activity data 141 includes the sensor data 143 and does not include any textual label data, provides the sensor data 143 to the textual label data generator 192 to generate textual label data 145. The textual label data 145 indicates a detected activity 181. In a particular example, the textual label data generator 192 provides the sensor data 143 to the speaker diarizer 112 in response to determining that the sensor data 143 includes speech data. The speaker diarizer 112 performs speaker diarization techniques to identify one or more portions of the sensor data 143 as corresponding to individual speakers. For example, the speaker diarizer 112 identifies audio data 171 of the sensor data 143 as corresponding to a first speaker, second audio data of the sensor data 143 as corresponding to a second speaker, or both.

In a particular example, the textual label data generator 192 provides the sensor data 143 to the user detector 114. The user detector 114 determines that the sensor data 143 is associated with the user 105 (associated with a user identifier (ID) 173) by performing facial recognition, voice recognition, thumbprint analysis, retinal analysis, user input, or a combination thereof. In a particular example, the user detector 114 determines that at least a portion of the sensor data 143 is associated with the user 105 by performing voice recognition on the audio data 171. The user detector 114, in response to determining that the sensor data 143 is associated with the user 105, generates (or updates) a detected activity 181 to indicate the user ID 173 of the user 105.

The textual label data generator 192, in response to determining that the sensor data 143 includes speech data and that the speaker diarizer 112 has identified the audio data 171 as corresponding to an individual speaker, provides the audio data 171 to the speech-to-text convertor 118. The speech-to-text convertor 118 generates user speech text 179 by performing speech recognition techniques to convert the audio data 171 to text. The speech-to-text convertor 118 generates (or updates) the detected activity 181 to indicate the user speech text 179.

The textual label data generator 192, in response to determining that the activity data 141 indicates the location 183, generates (or updates) the textual label data 145 to indicate the location 183. Alternatively, the textual label data generator 192, in response to determining that the activity data 141 does not indicate any location, provides the sensor data 143 to the location detector 120. In a particular aspect, the textual label data generator 192, in response to determining that the activity data 141 is received from the device 104, provides a device identifier of the device 104 to the location detector 120.

In a particular aspect, the location detector 120, in response to receiving the device identifier of the device 104, determines whether the device 104 is associated with a particular location. For example, the location detector 120 has access to location data indicating locations of various devices. In this example, the location detector 120, in response to determining that the location data indicates that the device 104 is associated with a location 183, updates the textual label data 145 to indicate the location 183.

In a particular aspect, the location detector 120, in response to receiving the sensor data 143, determines that the sensor data 143 is associated with the location 183. For example, the location detector 120 determines that the sensor data 143 is associated with the location 183 in response to determining that sensor data 143 includes coordinates, an address, or both, indicating the location 183. As another example, the location detector 120 determines that the sensor data 143 indicates the location 183 by performing image recognition on the sensor data 143 to determine that the sensor data 143 matches one or more images associated with the location 183. The location detector 120, in response to determining that the sensor data 143 is associated with the location 183, updates the textual label data 145 to indicate the location 183.

The textual label data generator 192 provides the sensor data 143 to the activity detector 122. The activity detector 122 is configured to determine whether the sensor data 143 indicates an event 163, a user action 161, or both. For example, the activity detector 122, in response to determining that the user detector 114 has detected the user 105 associated with the user ID 173, generates (or updates) the detected activity 181 to include the user action 161 indicating presence of the user 105. In another example, the activity detector 122 performs analysis (e.g., image analysis, audio analysis, text analysis, or a combination thereof) of the sensor data 143 to identify one or more movements performed by the user 105, and updates the detected activity 181 to include the user action 161 indicating the one or more movements (e.g., opening a door, changing a device setting, performing a gesture, playing a musical instrument, watching television, reading a book, exercising, eating, or a combination thereof). In a particular example, the activity detector 122 performs analysis (e.g., image analysis, audio analysis, text analysis, or a combination thereof) of the sensor data 143 to identify the event 163 (e.g., sound of breaking glass, activation of an alarm, a status update of the sensor(s) 142, or a combination thereof), and updates the detected activity 181 to indicate the event 163.

The textual label data generator 192 provides the sensor data 143, the audio data 171, the user action 161, the user speech text 179, or a combination thereof, to the emotion detector 116. The emotion detector 116 determines whether the sensor data 143 indicates any user emotion. For example, the emotion detector 116 detects a user emotion 175 by performing a voice analysis of the audio data 171, performing text analysis of the user speech text 179, performing action analysis (e.g., gesture analysis) of the user action 161, facial expression analysis of the sensor data 143 (e.g., image sensor data), or a combination thereof. The emotion detector 116, in response to detecting the user emotion 175, generates (or updates) the detected activity 181 to indicate the user emotion 175. The textual label data generator 192 generates (or updates) the textual label data 145 to indicate the detected activity 181, the location 183, the timestamp 177, or a combination thereof In a particular aspect, the textual label data 145 includes the timestamp 177 indicated by the activity data 141. In a particular aspect, the timestamp 177 of the textual label data 145 indicates a time at which the activity data 141 is received at the device 102, a time at which the textual label data 145 is generated, a time at which the textual label data 145 is stored in the memory 132, or a combination thereof.

The activity tracker 136, in response to determining that the activity data 141 includes the textual label data 145 or determining that the textual label data generator 192 has completed generating the textual label data 145, provides the textual label data 145 to the entry generator 194. The entry generator 194 generates an activity entry 111 in response to receiving the textual label data 145. In a particular aspect, the activity entry 111 includes the detected activity 181, the location 183, the timestamp 177, or a combination thereof, copied from the textual label data 145.

In a particular aspect, the entry generator 194 generates the activity entry 111 based on textual label data corresponding to activity data received from multiple devices. In a particular example, the activity data 141 from the device 104 (e.g., a microwave oven) indicates the user action 161 (e.g., opening a microwave oven) and the activity data 141 from the device 106 (e.g., a camera on top of the microwave oven) indicates the user identifier 173. In this example, the textual label data 145 associated with the device 104 indicates the user action 161 and second textual label data associated with the device 106 indicates the user ID 173. The entry generator 194 generates the activity entry 111 to indicate the user action 161 and the user ID 173 based on the textual label data 145 and the second textual label data, respectively.

The entry generator 194 generates (or updates) the activity log 107 to include the activity entry 111. In a particular aspect, the activity entry 111 is added in natural language to the activity log 107. For example, the entry generator 194 generates a sentence based on the detected activity 181, the location 183, the timestamp 177, or a combination thereof, and adds the sentence as the activity entry 111 to the activity log 107.

Authorized users can query the activity tracker 136. For example, a user 101 (e.g., an authorized user) provides a query 185 via the device 108 (e.g., a user device) to the device 102. In a particular aspect, the user 101 provides input (e.g., speech, typed input, or both) to the device 108 and the device 108 generates the query 185 indicating the input. In another example, the user 101 provides the input to the device 102 and the device 102 generates the query 185 based on the input. In a particular aspect, the query 185 includes a natural language query.

The query response system 138 generates a query response 187 in response to receiving the query 185. For example, the query response system 138 generates the query response 187 by performing an artificial intelligence analysis of the activity log 107 based on the query 185. In a particular aspect, the query response 187 includes an answer 191, a confidence score 189 associated with the answer 191, or both.

In a particular aspect, the query response system 138 generates the query response 187 by using a memory network architecture, a language model based on bidirectional encoder representations from transformers (BERT), a bi-directional attention flow (BiDAF) network, or a combination thereof. In a particular aspect, a memory network architecture includes an end-to-end memory network. For example, the query response system 138 generates the query response 187 by using a neural network with a recurrent attention model that is trained end-to-end. To illustrate, during training, the neural network is provided a training activity log. The entries of the training activity log are converted into memory vectors by embedding each activity entry into a first embedding matrix. A training query is embedded into a second embedding matrix. Each entry has a corresponding output vector (e.g., represented by a third embedding matrix). A predicted answer is generated based on the second embedding matrix, the third embedding matrix, and a weight matrix. During training of the neural network, the first embedding matrix, the second embedding matrix, the third embedding matrix, and the weight matrix are trained based on a comparison of predicted answers and training answers. The trained neural network is used to generate the answer 191 using the activity log 107 and the query 185. In a particular aspect, the query response system 138 uses the trained neural network to generate the confidence score 189 of the answer 191.

In a particular aspect, the query response system 138 generates the query response 187 by using a language model based on BERT. In a particular aspect, a BERT architecture includes a multi-layer bidirectional transformer encoder. In a particular aspect, the language model is trained using a masked language model (MLM). The query response system 138 uses the trained language model to identify a portion of the activity log 107 as an answer 191 for the query 185. In a particular aspect, the query response system 138 uses the trained language model to generate the confidence score 189 of the answer 191.

In a particular aspect, the query response system 138 generates the query response 187 by using a BiDAF network. In a particular aspect, a BiDAF network includes a hierarchical multi-stage architecture for modeling representations of context at different levels of granularity. BiDAF includes character-level, word-level, and phrase-level embeddings, and uses bi-directional attention flow for query-aware context representation. In a particular aspect, the BiDAF computes an attention vector for every time step and the attention vector, along with representations from previous layers, is allowed to flow through to subsequent modelling layers. The query response system 138 uses the trained BiDAF network to identify a portion of the activity log 107 as an answer 191 for the query 185. In a particular aspect, the query response system 138 uses the trained BiDAF to generate the confidence score 189 of the answer 191.

In a particular aspect, the query response system 138 provides the query response 187 to the device 108. Alternatively, or in addition, the query response system 138 provides the query response 187 to a display device coupled to the device 102.

In a particular aspect, the device 102, the device 104, the device 106, the device 108, or a combination thereof, are included in a vehicle (e.g., a car). In a particular example, the sensor(s) 142 generate the sensor data 143 indicating that various people entered or exited the vehicle at different times, that seats of the vehicle were occupied by various people at particular times, that the vehicle travelled to particular locations, that particular operations were performed by particular people at particular times, or a combination thereof. To illustrate, the sensor(s) 142 generate the sensor data 143 including image data indicating that the user 105 occupied the driver's seat of the vehicle at a particular time, image data indicating that the user 103 occupied a passenger seat of the vehicle, sensor data indicating that the user 103 increased a volume of a music player of the vehicle, location data indicating a particular location of the vehicle, and vehicle status data indicating that the vehicle was travelling at a particular speed at the particular time. The activity log 107 includes entries indicating that the user 105 occupied the driver seat, that the user 103 occupied the passenger seat, that the user 103 increased the music volume, that the vehicle traveled to the particular location (e.g., a geographical location, a particular store, a gas station, a supermarket, or a combination thereof), that the vehicle was operating at the particular speed at the particular time, or a combination thereof In this example, a user 101 (e.g., a vehicle owner, such as a parent or an employer) can send a query 185 to the query response system 138 requesting information regarding the vehicle (e.g., “what speed was the user 105 driving the vehicle?”). The query response system 138 generates a query response 187 (e.g., indicating the particular speed) by analyzing the activity log 107 based on the query 185 and provides the query response 187 to a display, the device 108, or both.

A text-based activity log (e.g., the activity log 107) combined with artificial intelligence techniques (e.g., machine learning) of the query response system 138 enables generating query responses (e.g., the query response 187) for natural language queries (e.g., the query 185) using relatively few processing and memory resources. Using fewer processing and memory resources enables the activity tracking and query response generation to be performed locally to increase privacy, as compared to cloud-based processing of activity data (e.g., the activity data 141, the activity log 107, or both).

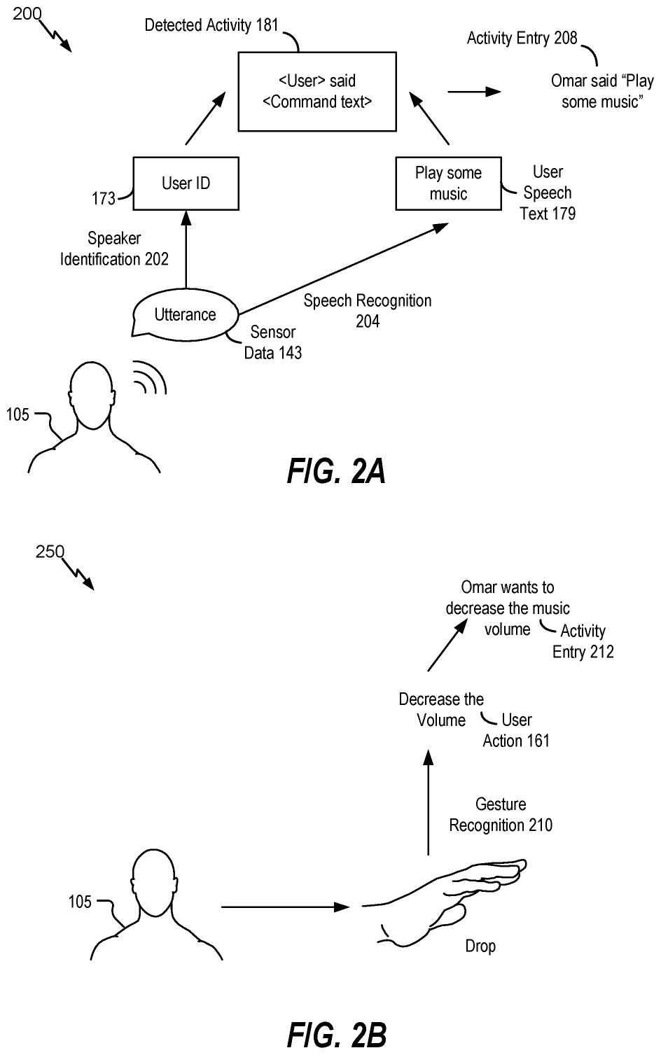

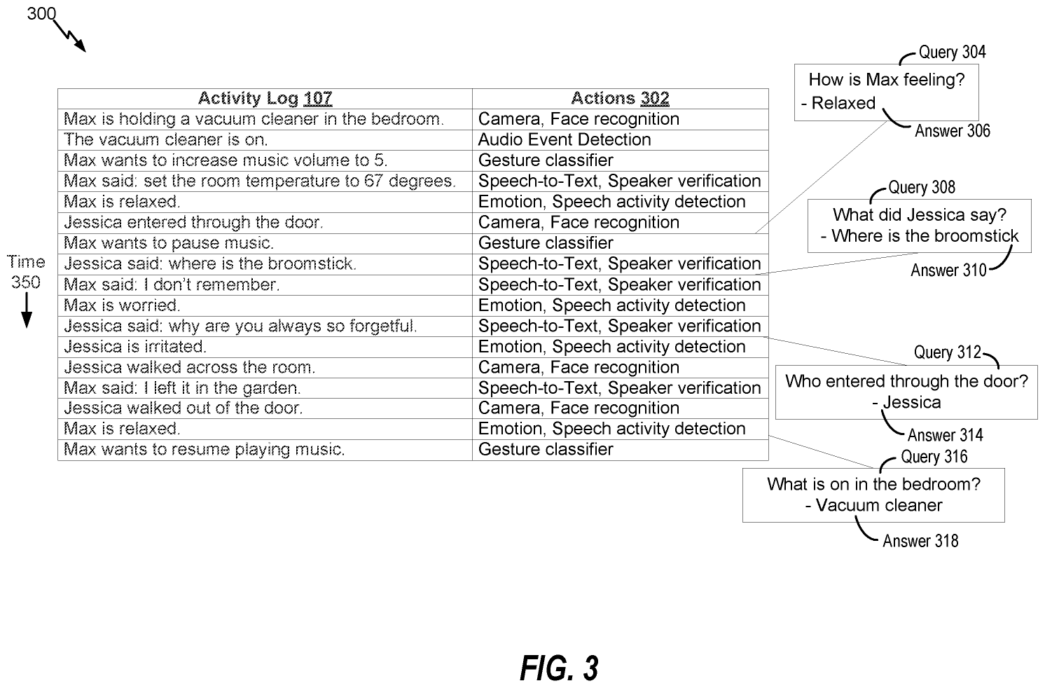

Examples of activity detection are provided in FIGS. 2A-2B. Examples of activity logs and queries are provided in FIGS. 3-5.

Referring to FIG. 2A, an example of activity detection is shown and generally designated 200. In a particular aspect, the activity detection 200 is performed by the speaker diarizer 112, the user detector 114, the speech-to-text convertor 118, the textual label data generator 192, the entry generator 194, the activity detector 122, the activity tracker 136, the processor(s) 110, the interface 134, the device 102, the sensor(s) 142, the device 104, the system 100 of FIG. 1, or a combination thereof.

An utterance of the user 105 is detected by the sensor(s) 142 of the device 104 of FIG. 1. For example, the user 105 speaks (e.g., “Play some music”) within a coverage area of the sensor(s) 142 (e.g., a microphone). The sensor(s) 142 generate sensor data 143 (e.g., the audio data 171) corresponding to the utterance of the user 105.

In a particular aspect, the device 104 provides the sensor data 143 as the activity data 141 to the device 102. In this aspect, the textual label data generator 192 of the device 102 generates textual label data 145 based on the sensor data 143 received as the activity data 141. In an alternate aspect, the device 104 performs one or more operations described herein with reference to the textual label data generator 192 to generate textual label data 145. In this aspect, the device 104 provides the textual label data 145 to the device 102 as the activity data 141.

The activity detection 200 includes performing speaker identification 202. For example, the user detector 114 of FIG. 1 determines that the sensor data 143 corresponds to the user 105 associated with the user ID 173, as described with reference to FIG. 1. The user detector 114 generates (or updates) textual label data 145 associated with the sensor data 143 to indicate the user ID 173.

The activity detection 200 includes performing speech recognition 204. For example, the speech-to-text convertor 118 of FIG. 1 generates the user speech text 179 (e.g., “play some music”) by performing speech recognition on the sensor data 143, as described with reference to FIG. 1. The speech-to-text convertor 118 generates (or updates) the textual label data 145 to indicate the user speech text 179. A detected activity 181 of the textual label data 145 including the user ID 173 and the user speech text 179 indicates that the user 105 said words indicated in the user speech text 179 (e.g., “play some music”).

The entry generator 194 of FIG. 1 generates an activity entry 208 based on the textual label data 145. For example, the entry generator 194, in response to determining that the detected activity 181 of the textual label data 145 indicates that the user 105 said the words indicated by the user speech text 179, generates the activity entry 208 to indicate that the user 105 said the words indicated by the user speech text 179. In a particular aspect, the activity entry 208 includes natural language. For example, the activity entry 208 includes one or more sentences (e.g., “Omar said ‘play some music’”) in a natural language (e.g., English, Spanish, French, Arabic, Hindi, Chinese, Urdu, Tamil, etc.). For example, the entry generator 194 generates the activity entry 208 (e.g., “Omar said ‘play some music’.”) in English to include a name (e.g., “Omar”) of the user 105 followed by the word “said” followed by the user speech text 179 in quotes (e.g., “‘play some music’”) followed by a period (e.g., “.”). In some examples, the activity entry 208 does not include a period. The entry generator 194 adds the activity entry 208 to the activity log 107. In a particular aspect, the activity entry 208 corresponds to the activity entry 111 of FIG. 1.

The activity detection 200 thus illustrates that information (e.g., the user ID 173 and the user speech text 179) generated by performing separate analysis (e.g., the speaker identification 202 and the speech recognition 204) of the sensor data 143 can be combined to generate the activity entry 208.

Referring to FIG. 2B, an example of activity detection is shown and generally designated 250. In a particular aspect, the activity detection 250 is performed by the user detector 114, the activity detector 122, the textual label data generator 192, the entry generator 194, the activity tracker 136, the processor(s) 110, the interface 134, the device 102, the sensor(s) 142, the device 104, the system 100 of FIG. 1, or a combination thereof.

The user 105 is detected by the sensor(s) 142 of the device 104 of FIG. 1. For example, the sensor(s) 142 (e.g., a camera) capture image data (e.g., a video or a plurality of images) of the user 105. The sensor(s) 142 generate sensor data 143 including the image data.

In a particular aspect, the device 104 provides the sensor data 143 as the activity data 141 to the device 102. In this aspect, the textual label data generator 192 of the device 102 generates textual label data 145 based on the sensor data 143 received as the activity data 141. In an alternate aspect, the device 104 performs one or more operations described herein with reference to the textual label data generator 192 to generate textual label data 145. In this aspect, the device 104 provides the textual label data 145 to the device 102 as the activity data 141.

In a particular example, the user detector 114 of FIG. 1 determines that the sensor data 143 corresponds to the user 105 associated with the user ID 173, as described with reference to FIG. 1. For example, the user detector 114 performs facial recognition on the image data to determine that the sensor data 143 corresponds to the user 105. The user detector 114 generates (or updates) textual label data 145 associated with the sensor data 143 to indicate the user ID 173 of the user 105.

The activity detection 250 includes performing gesture recognition 210. For example, the activity detector 122 of FIG. 1 generates a user action 161 (e.g., hand drop gesture) by performing gesture recognition on the sensor data 143 (e.g., the image data or motion data of a wearable electronic device, such as a ‘smart watch’). In a particular aspect, the activity detector 122 performs gesture recognition to determine whether the sensor data 143 indicates any of a predetermined set of gestures (e.g., a hand drop gesture, a hand raise gesture, a hand stop gesture, a left-to-right swipe gesture, or a right-to-left swipe gesture) associated with a type (e.g., a music playback device) of the device 104 that generated the sensor data 143. In a particular aspect, device type data associated with type of the device 104 indicates the predetermined set of gestures. In a particular aspect, the device type data is based on default data, configuration data, user input, or a combination thereof.

In a particular aspect, the activity detector 122, in response to determining that the sensor data 143 indicates one or more gestures, determines a user action 161 based on the device 104. For example, the activity detector 122, in response to determining that the sensor data 143 indicates one or more gestures, determines whether the one or more gestures are included in a predetermined set of gestures associated with the device 104. The activity detector 122, in response to determining that the one or more gestures are included in the predetermined set of gestures associated with the device 104, determines whether the device type data indicates any user action associated with the one or more gestures. In a particular aspect, the activity detector 122 determines that the device type data indicates that a particular user action (e.g., “decrease the volume”) is associated with the one or more gestures (e.g., a hand drop gesture). The activity detector 122 adds the particular user action as the user action 161 to the textual label data 145.

The entry generator 194 of FIG. 1 generates an activity entry 212 based on the textual label data 145. For example, the entry generator 194, in response to determining that the textual label data 145 indicates the user ID 173 of the user 105 and the user action 161, generates the activity entry 212 indicating that the user 105 wants to perform or performed the user action 161 (e.g., “Omar wants to decrease the volume.”). In a particular aspect, the entry generator 194 generates the activity entry 212 based on a context (e.g., a previous entry) indicated by the activity log 107. For example, the entry generator 194, in response to determining that the activity log 107 includes the activity entry 208 indicating a previous interaction with the device 104 (e.g., “Play some music”), generates the activity entry 212 (e.g., “Omar wants to decrease the music volume.”) based on the user ID 173, the user action 161, and the activity entry 208. The entry generator 194 adds the activity entry 212 to the activity log 107. In a particular aspect, the activity entry 212 corresponds to the activity entry 111 of FIG. 1. The activity detection 250 thus illustrates an example of generating an activity entry based on non-audio sensor data (e.g., data indicating a user gesture).

Referring to FIG. 3, a diagram 300 illustrates examples of the activity log 107, actions 302 performed by the activity tracker 136 to generate corresponding entries of the activity log 107, queries received by the query response system 138, and corresponding answers generated by the query response system 138.

The actions 302 are performed in sequence along a time axis 350. The activity log 107 is updated in sequence along the time axis 350. In a particular aspect, the query response system 138 receives queries during or between updates of the activity log 107. For example, a query 304 (e.g., “How is Max feeling?”) is received by the query response system 138 subsequent to adding a first entry (e.g., “Jessica entered through the door.”) to the activity log 107 and prior to adding a second entry (e.g., “Max wants to pause music.”) to the activity log 107. The query response system 138 generates an answer 306 based on entries that are added to the activity log 107 prior to receiving the query 304. For example, the query response system 138 uses artificial intelligence techniques to generate the answer 306 (e.g., “Relaxed”), as described with reference to FIG. 1. In a particular aspect, the query response system 138, in response to determining that the query 304 indicates a particular user (e.g., “Max”) and is requesting a user emotion (e.g., “How” and “feeling”) and that a most recent entry (e.g., “Max is relaxed”) of the activity log 107 that is associated with the particular user indicates a particular user emotion (e.g., “relaxed”), generates the answer 306 indicating the particular user emotion (e.g., “relaxed”).

A query 308 (e.g., “What did Jessica say?”) indicates a particular user (e.g., “Jessica”) and is requesting user speech text (e.g., “What” and “say”). The query response system 138, in response to determining that a most recent entry (e.g., “Jessica said: where is the broomstick.”) of the activity log 107 that indicates the particular user (e.g., “Jessica”) and particular user speech text (e.g., “Where is the broomstick”), generates an answer 310 (e.g., “Where is the broomstick”) indicating the particular user speech text.

A query 312 (e.g., “Who entered through the door?”) indicates a particular user action (e.g., “entered through the door”) and is requesting an actor (e.g., “Who”) that performed the particular user action. The query response system 138, in response to determining that a most recent entry (e.g., “Jessica entered through the door”) of the activity log 107 that indicates the particular user action (e.g., “entered through the door”) indicates a particular user (e.g., “Jessica”), generates an answer 314 (e.g., “Jessica”) that indicates the particular user.

A query 316 (e.g., “What is on in the bedroom?”) indicates a state (e.g., “on”) and a particular location (e.g., “bedroom”) and is requesting an actor (e.g., “What”) that is in the state. The query response system 138, in response to determining that a most recent entry (e.g., “The vacuum cleaner is on.”) of the activity log 107 that indicates the particular location (e.g., “bedroom”) and the state (e.g., “on”) indicates a particular actor (e.g., “vacuum cleaner”), generates an answer 318 (e.g., “Vacuum cleaner”) indicating the particular actor.

The diagram 300 thus illustrates queries requesting various types of activity data using natural language. The query response system 138 generates answers to the queries by analyzing the activity log 107 based on the queries.

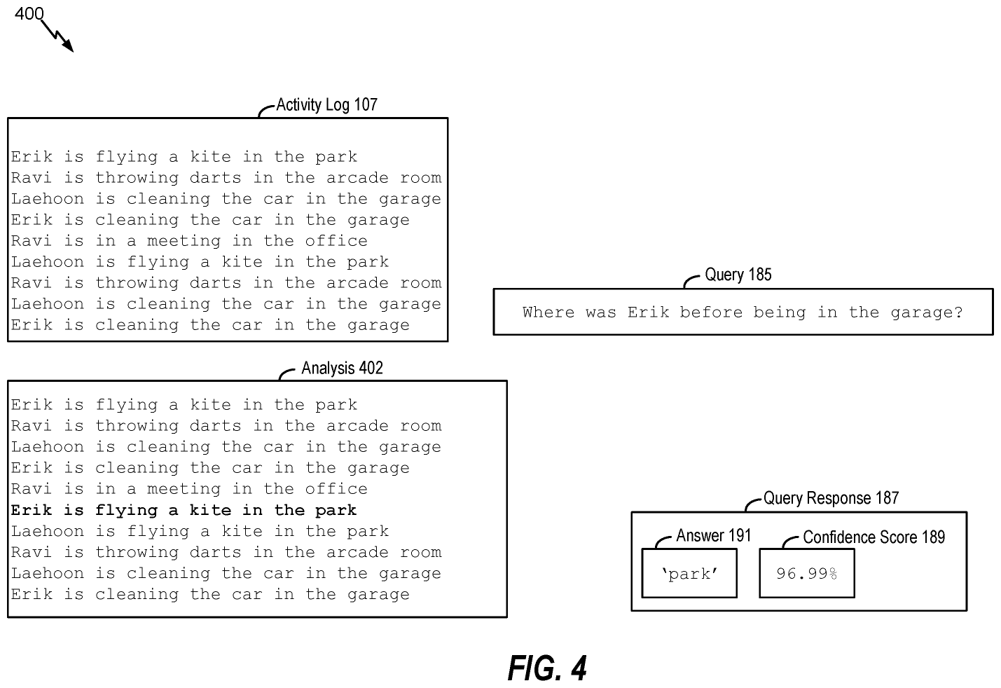

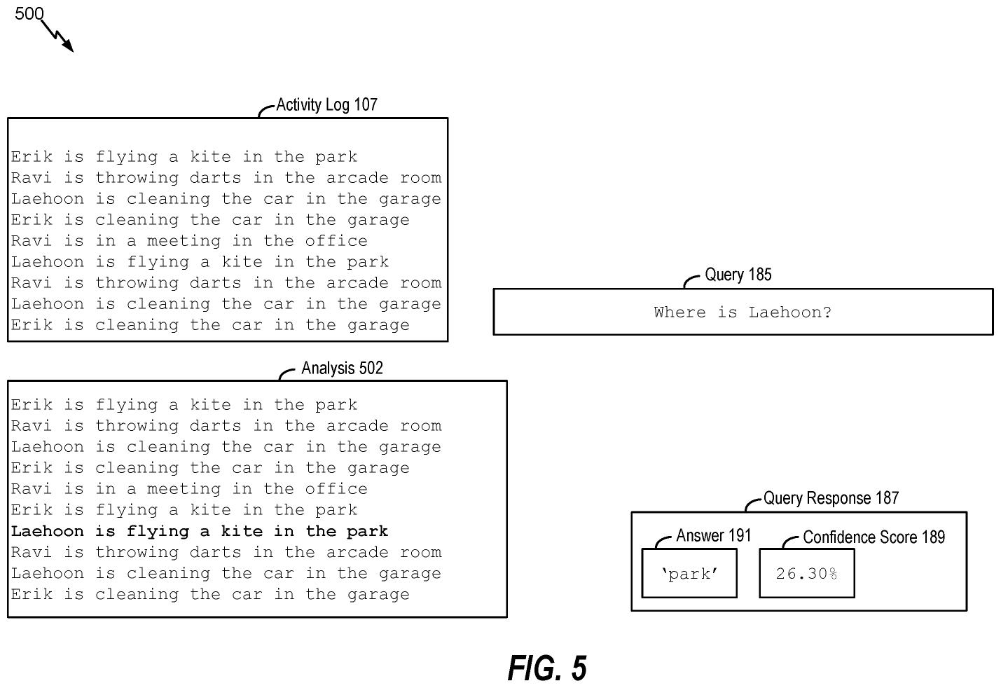

Although, the description of FIG. 3 (and of FIGS. 4-5 below) provide examples of searching an activity log based on parsing a query for locations, users, states, etc., for clarity of explanation, it should be understood that an artificial intelligence (or machine learning) implementation of the query response system 138 may not parse queries for such identifying information and may instead apply any other type of analysis of a query and an activity log to determine an answer.

Referring to FIG. 4, a diagram 400 illustrates examples of an activity log 107, a query 185, an analysis 402 of the activity log 107, and a query response 187. In a particular aspect, the query response system 138 of FIG. 1 receives a query 185 (e.g., “Where was Erik before being in the garage?”) and performs an analysis 402 to generate a query response 187. For example, the query response system 138 generates the query response 187 by using artificial intelligence techniques to analyze the activity log 107 based on the query 185, as described with reference to FIG. 1.

In a particular aspect, the query 185 (e.g., “Where was Erik before being in the garage?”) indicates a particular user (e.g., “Erik”) and a particular location (e.g., “garage”) and is requesting a location (e.g., “Where”) of the particular user prior to (e.g., “before”) being in the particular location. In a particular aspect, the query response system 138 performs an analysis 402 of the activity log 107 to identify a first most recent entry (e.g., “Erik is cleaning the car in the garage”) of the activity log 107 that indicates the particular user (e.g., “Erik”) in the particular location (e.g., “garage”). The query response system 138 performs the analysis 402 to identify a second most recent entry (e.g., “Erik is flying a kite in the park”) prior to the first most recent entry (e.g., “Erik is cleaning the car in the garage”) in the activity log 107 that indicates the particular user (e.g., “Erik”) and a second location (e.g., “park”) that is distinct from the particular location (e.g., “garage”). The query response system 138 generates an answer 191 indicating the second location (e.g., “park”). In a particular aspect, the query response system 138 determines, based on the artificial intelligence techniques, a confidence score 189 (e.g., 96.99%) associated with the answer 191. The query response system 138 generates a query response 187 indicating the answer 191, the confidence score 189, or both.

The diagram 400 thus illustrates that the query response system 138 can generate query responses for queries that request activity information related to (e.g., prior to or subsequent to) other activity information.

Referring to FIG. 5, a diagram 500 illustrates examples of an activity log 107, a query 185, an analysis 502 of the activity log 107, and a query response 187. In a particular aspect, the query response system 138 of FIG. 1 receives a query 185 (e.g., “Where is Laehoon?”) and performs an analysis 502 to generate a query response 187. For example, the query response system 138 generates the query response 187 by using artificial intelligence techniques to analyze the activity log 107 based on the query 185, as described with reference to FIG. 1.

In a particular aspect, the query 185 (e.g., “Where is Laehoon?”) indicates a particular user (e.g., “Laehoon”) and requests a location (e.g., “Where”) of the particular user. In a particular aspect, the query response system 138 performs an analysis 502 of the activity log 107 to identify an entry (e.g., “Laehoon is flying a kite in the park”) of the activity log 107 that indicates the particular user (e.g., “Laehoon”) and a particular location (e.g., “park”). The query response system 138 generates an answer 191 indicating the particular location (e.g., “park”). In a particular aspect, the query response system 138 determines, based on the artificial intelligence techniques, a confidence score 189 (e.g., 26.30%) associated with the answer 191. In a particular aspect, the confidence score 189 (e.g., 26.30%) is relatively low (e.g., lower than 50%) because the activity log 107 includes multiple entries indicating the particular user (e.g., “Laehoon”) and various locations. For example, the activity log 107 includes a second entry (e.g., “Laehoon is cleaning the car in the garage”) indicating the particular user (e.g., “Laehoon”) and a second location (e.g., “garage”). The query response system 138 generates a query response 187 indicating the answer 191, the confidence score 189, or both. In a particular aspect, the confidence score 189 indicates a reliability of the answer 191 to the user 101. In a particular aspect, the query response system 138 generates the query response 187 including multiple answers and corresponding confidence scores. In this aspect, the query response 187 includes the answer 191 (e.g., “park”) and a second answer (e.g., “garage”) along with the confidence score 189 of the answer 191 and a second confidence score of the second answer. The diagram 500 thus illustrates that the query response system 138 can generate query responses that indicate reliability of the answers provided in the query responses.

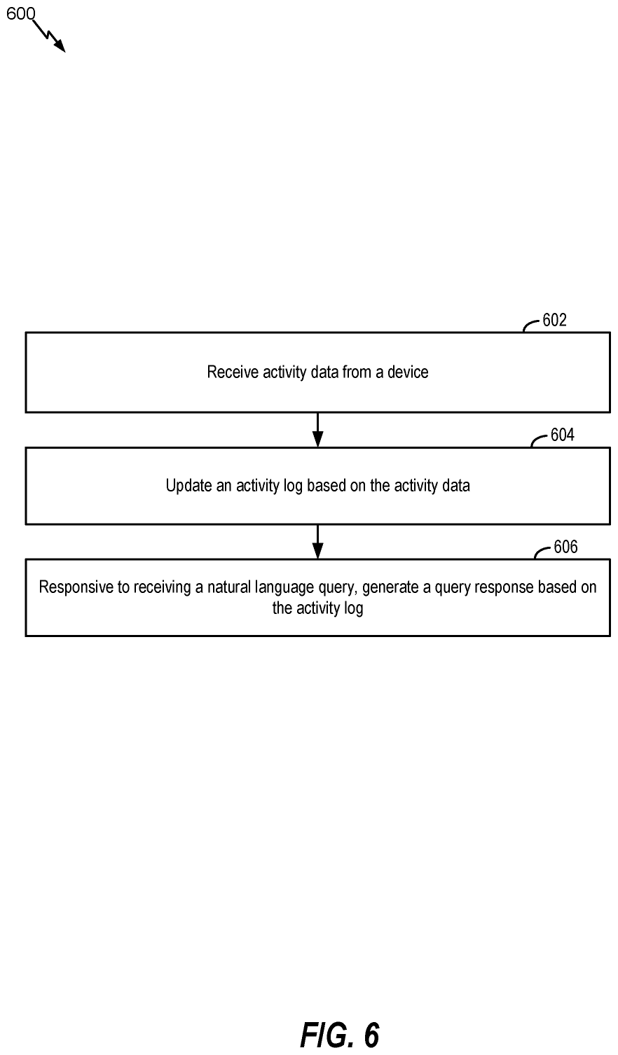

Referring to FIG. 6, a method of activity query response generation is shown and generally designated 600. In a particular aspect, one or more operations of the method 600 are performed by the interface 134, the entry generator 194, the speaker diarizer 112, the user detector 114, the activity detector 122, the speech-to-text convertor 118, the location detector 120, the emotion detector 116, the textual label data generator 192, the activity tracker 136, the query response system 138, the processor(s) 110, the device 102, the device 104, the system 100 of FIG. 1, or a combination thereof

The method 600 includes receiving activity data from a device, at 602. For example, the interface 134 of FIG. 1 receives the activity data 141 from the device 104, as described with reference to FIG. 1.

The method 600 also includes updating an activity log based on the activity data, at 604. For example, the entry generator 194 of FIG. 1 updates the activity log 107 based on the activity data 141, as described with reference to FIG. 1.

The method 600 further includes, responsive to receiving a natural language query, generating a query response based on the activity log, at 606. For example, the query response system 138 of FIG. 1, responsive to receiving a query 185 (e.g., a natural language query), generates a query response 187 based on the activity log 107, as described with reference to FIG. 1.

The method 600 thus enables updating an activity log to track activities indicated by activity. The method 600 also enables generating query responses based on the activity log for natural language queries.

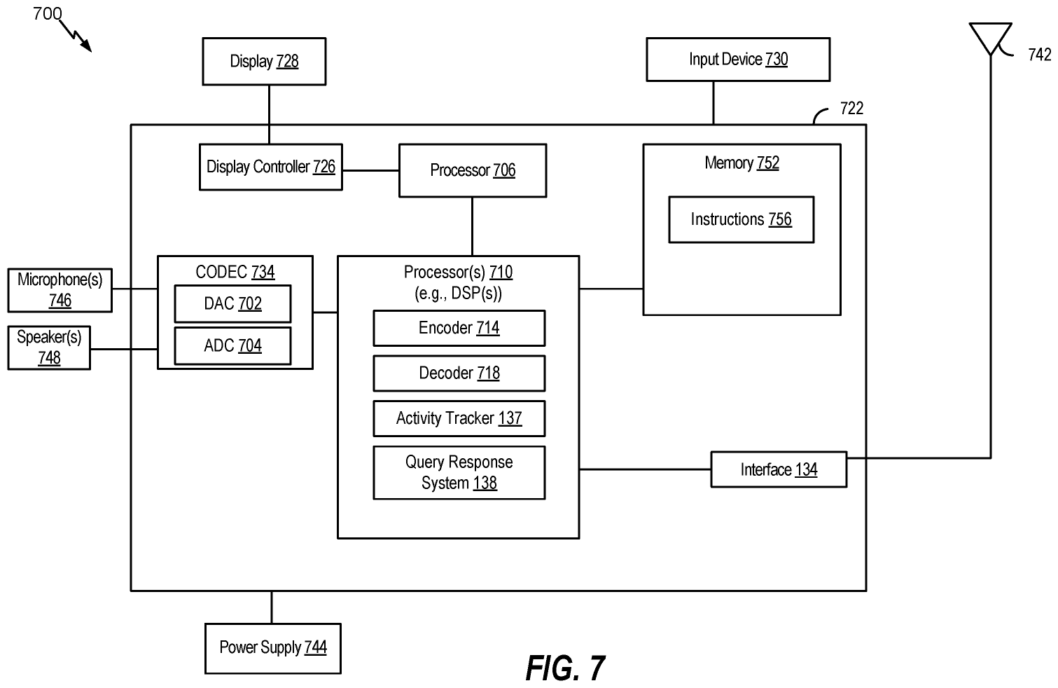

Referring to FIG. 7, a block diagram of a particular illustrative example of a device (e.g., a wireless communication device) is depicted and generally designated 700. In various aspects, the device 700 may have fewer or more components than illustrated in FIG. 7. In an illustrative aspect, the device 700 may correspond to the device 102, the device 104, the device 106, the device 108 of FIG. 1, or a combination thereof. In an illustrative aspect, the device 700 may perform one or more operations described with reference to systems and methods of FIGS. 1-6.

In a particular aspect, the device 700 includes a processor 706 (e.g., a central processing unit (CPU)). The device 700 may include one or more additional processors 710 (e.g., one or more digital signal processors (DSPs)). In a particular aspect, the processors 710 correspond to the processor(s) 110 of FIG. 1. The processors 710 may include a decoder 718, an encoder 714, one or more components of the activity tracker 136, the query response system 138, or a combination thereof.

The device 700 may include a memory 752 and a CODEC 734. Although the encoder 714, the decoder 718, the activity tracker 136, and the query response system 138 are illustrated as components of the processors 710 (e.g., dedicated circuitry and/or executable programming code), in other aspects one or more components of the encoder 714, the decoder 718, the activity tracker 136, the query response system 138, or a combination thereof may be included in the processor 706, the CODEC 734, another processing component, or a combination thereof.

The device 700 may include the interface 134 coupled to one or more antennas 742. The processors 710 may be coupled to the interface 134. The device 700 may include a display 728 coupled to a display controller 726. One or more speakers 748 (e.g., loudspeakers) may be coupled to the CODEC 734. One or more microphones 746 may be coupled, via one or more input interface(s), to the CODEC 734. The CODEC 734 may include a digital-to-analog converter (DAC) 702 and an analog-to-digital converter (ADC) 704.

The memory 752 may include instructions 756 executable by the processor 706, the processors 710, the CODEC 734, another processing unit of the device 700, or a combination thereof, to perform one or more operations described with reference to FIGS. 1-6. The memory 752 may store one or more signals, one or more parameters, one or more thresholds, one or more indicators, or a combination thereof, described with reference to FIGS. 1-6. In a particular aspect, the memory 752 corresponds to the memory 132 of FIG. 1.

One or more components of the device 700 may be implemented via dedicated hardware (e.g., circuitry), by a processor executing instructions to perform one or more tasks, or a combination thereof. As an example, the memory 752 or one or more components of the processor 706, the processors 710, and/or the CODEC 734 may be a memory device (e.g., a computer-readable storage device), such as a random access memory (RAM), magnetoresistive random access memory (MRAM), spin-torque transfer MRAM (STT-MRAM), flash memory, read-only memory (ROM), programmable read-only memory (PROM), erasable programmable read-only memory (EPROM), electrically erasable programmable read-only memory (EEPROM), registers, hard disk, a removable disk, or a compact disc read-only memory (CD-ROM). The memory device may include (e.g., store) instructions (e.g., the instructions 756) that, when executed by a computer (e.g., one or more processors, such as a processor in the CODEC 734, the processor 706, and/or the processors 710), may cause the computer to perform one or more operations described with reference to FIGS. 1-6. As an example, the memory 752 or the one or more components of the processor 706, the processors 710, and/or the CODEC 734 may be a non-transitory computer-readable medium that includes instructions (e.g., the instructions 756) that, when executed by a computer (e.g., one or more processors, such as a processor in the CODEC 734, the processor 706, and/or the processors 710), cause the computer perform one or more operations described with reference to FIGS. 1-6.

In a particular aspect, the device 700 may be included in a system-in-package or system-on-chip device (e.g., a mobile station modem (MSM)) 722. In a particular aspect, the processor 706, the processors 710, the display controller 726, the memory 752, the CODEC 734, and the interface 134 are included in a system-in-package or the system-on-chip device 722. In a particular aspect, an input device 730, such as an image sensor, a touchscreen, and/or keypad, and a power supply 744 are coupled to the system-on-chip device 722. Moreover, in a particular aspect, as illustrated in FIG. 7, the display 728, the input device 730, the speaker(s) 748, the microphone(s) 746, the antenna(s) 742, and the power supply 744 are external to the system-on-chip device 722. However, each of the display 728, the input device 730, the speaker(s) 748, the microphone(s) 746, the antenna(s) 742, and the power supply 744 can be coupled to a component of the system-on-chip device 722, such as an interface or a controller.

In a particular aspect, the microphone(s) 746 are configured to receive the query 185 of FIG. 1 as audio data. In a particular aspect, the antenna(s) 742 are configured to receive the activity data 141 of FIG. 1 from the device 104. In a particular aspect, the speaker(s) 748 are configured to output the query response 187 of FIG. 1 as audio. In a particular aspect, the input device 730 includes one or more image sensors that are configured to receive the query 185 as image data.

The device 700 may include a home appliance, an IoT device, an IoT device controller, factory equipment, a security system, a wireless telephone, a mobile communication device, a mobile device, a mobile phone, a smart phone, a cellular phone, a virtual reality headset, an augmented reality headset, a vehicle (e.g., a car), a laptop computer, a desktop computer, a computer, a tablet computer, a set top box, a personal digital assistant (PDA), a display device, a television, a gaming console, a music player, a radio, a video player, an entertainment unit, a communication device, a fixed location data unit, a personal media player, a digital video player, a digital video disc (DVD) player, a tuner, a camera, a navigation device, a decoder system, an encoder system, or any combination thereof.

In a particular aspect, one or more components of the systems described with reference to FIGS. 1-6 and the device 700 may be integrated into a decoding system or apparatus (e.g., an electronic device, a CODEC, or a processor therein), into an encoding system or apparatus, or both. In other aspects, one or more components of the systems described with reference to FIGS. 1-6 and the device 700 may be integrated into home appliance, an IoT device, an IoT device controller, factory equipment, a security system, a mobile device, a wireless telephone, a tablet computer, a desktop computer, a virtual reality headset, an augmented reality headset, a vehicle (e.g., a car), a laptop computer, a set top box, a music player, a video player, an entertainment unit, a television, a game console, a navigation device, a communication device, a personal digital assistant (PDA), a fixed location data unit, a personal media player, or another type of device.

It should be noted that various functions performed by the one or more components of the systems described with reference to FIGS. 1-6 and the device 700 are described as being performed by certain components or modules. This division of components and modules is for illustration only. In an alternate aspect, a function performed by a particular component or module may be divided amongst multiple components or modules. Moreover, in an alternate aspect, two or more components or modules described with reference to FIGS. 1-6 may be integrated into a single component or module. Each component or module described with reference to FIGS. 1-6 may be implemented using hardware (e.g., a field-programmable gate array (FPGA) device, an application-specific integrated circuit (ASIC), a DSP, a controller, etc.), software (e.g., instructions executable by a processor), or any combination thereof.

In conjunction with the described aspects, an apparatus includes means for storing an activity log. For example, the means for storing an activity log may include the memory 132, the device 102, the system 100 of FIG. 1, the memory 752, the device 700 of FIG. 7, one or more structures, devices, or circuits configured to store an activity log, or a combination thereof.

The apparatus also includes means for updating the activity log based on activity data. For example, the means for updating the activity log include the entry generator 194, the activity tracker 136, the processor(s) 110, the device 102, the system 100 of FIG. 1, the processor 706, the processors 710, the device 700 of FIG. 7, one or more structures, devices, or circuits configured to update an activity log based on activity data, or a combination thereof. The activity data is received from a second device (e.g., the device 104 of FIG. 1).

The apparatus further includes means for generating a query response based on the activity log. For example, the means for generating a query response include the query response system 138, the processor(s) 110, the device 102, the system 100 of FIG. 1, the processor 706, the processors 710, the device 700 of FIG. 7, one or more structures, devices, or circuits configured to generate a query response based on an activity log, or a combination thereof. The query response is generated responsive to receiving a natural language query (e.g., the query 185 of FIG. 1).

The previous description of the disclosed aspects is provided to enable a person skilled in the art to make or use the disclosed aspects. Various modifications to these aspects will be readily apparent to those skilled in the art, and the principles defined herein may be applied to other aspects without departing from the scope of the disclosure. Thus, the present disclosure is not intended to be limited to the aspects shown herein but is to be accorded the widest scope possible consistent with the principles and novel features as defined by the following claims.

What is claimed is:

1. A device for activity tracking comprising:
a memory configured to store an activity log; and
one or more processors configured to:
update the activity log based on activity data, the activity data received from a second device; and
responsive to receiving a natural language query, generate a query response based on the activity log.

2. The device of claim 1, further comprising an interface configured to receive the activity data from the second device.

3. The device of claim 1, further comprising an interface configured to enable local wireless networking with one or more local devices and to isolate the device from external networks, and wherein the one or more local devices include the second device.

4. The device of claim 1, further comprising an interface configured to receive the natural language query from a portable electronic device.

5. The device of claim 1, wherein the one or more processors are configured to:
determine, based on the activity data, a time and a location of a detected activity; and
update the activity log by adding an entry to the activity log, the entry indicating the time and the location of the detected activity.

6. The device of claim 5, wherein the detected activity indicates at least one of user emotion, user speech, user identification, or user action.

7. The device of claim 5, wherein the activity data includes textual label data that indicates the location, the detected activity, or both.

8. The device of claim 5, wherein the activity data includes sensor data, and wherein the one or more processors are configured to determine, based on the sensor data, the location, the detected activity, or both.

9. The device of claim 1, wherein the one or more processors are configured to generate the query response using a memory network architecture, a language model based on bidirectional encoder representations from transformers (BERT), a bi-directional attention flow (BiDAF) network, or a combination thereof.

10. The device of claim 1, further comprising a display configured to display the query response.

11. The device of claim 1, further comprising one or more microphones configured to receive the natural language query as audio data.

12. The device of claim 1, further comprising one or more antennas configured to receive the activity data from the second device.

13. The device of claim 1, further comprising one or more loudspeakers configured to output the query response as audio.

14. The device of claim 1, further comprising one or more image sensors configured to receive the natural language query as image data.

15. The device of claim 1, wherein at least one of the memory, the one or more processors, or a combination thereof are integrated into a vehicle.

16. A method of activity tracking, the method comprising:
receiving activity data at a first device from a second device;
updating, at the first device, an activity log based on the activity data; and
responsive to receiving a natural language query, generating a query response based on the activity log.

17. The method of claim 16, further comprising providing the query response to a display.

18. The method of claim 16, further comprising providing the query response to a user device, wherein the natural language query is received from the user device.

19. The method of claim 16, further comprising enabling local wireless networking with one or more local devices and isolating the first device from external networks, wherein the one or more local devices include the second device.

20. The method of claim 16, further comprising:
determining, based on the activity data, a time and a location of a detected activity; and
updating the activity log by adding an entry to the activity log, the entry indicating the time and the location of the detected activity.

21. The method of claim 20, wherein the detected activity indicates at least one of user emotion, user speech, user identification, or user action.

22. The method of claim 20, further comprising determining, based on sensor data, the location, the detected activity, or both, wherein the activity data includes the sensor data.

23. A computer-readable storage device storing instructions that, when executed by one or more processors, cause the one or more processors to:
update an activity log based on activity data received from a device; and
responsive to receiving a natural language query, generate a query response based on the activity log.

24. The computer-readable storage device of claim 23, wherein the instructions, when executed by the one or more processors, cause the one or more processors to:
determine, based on the activity data, a time and a location of a detected activity; and
update the activity log by adding an entry to the activity log, the entry indicating the time and the location of the detected activity.

25. The computer-readable storage device of claim 24, wherein the activity data includes textual label data that indicates the location, the detected activity, or both.

26. The computer-readable storage device of claim 24, wherein the instructions, when executed by the one or more processors, cause the one or more processors to determine, based on sensor data, the location, the detected activity, or both, wherein the activity data includes the sensor data.

27. The computer-readable storage device of claim 23, wherein the instructions, when executed by the one or more processors, cause the one or more processors to generate the query response using a memory network architecture, a language model based on bidirectional encoder representations from transformers (BERT), or both.

28. The computer-readable storage device of claim 23, wherein the instructions, when executed by the one or more processors, cause the one or more processors to generate the query response using a bi-directional attention flow (BiDAF) network.

29. An apparatus for activity tracking comprising:
means for storing an activity log; and
means for updating the activity log based on activity data, the activity data received from a second device; and
means for generating a query response based on the activity log, the query response generated responsive to receiving a natural language query.

30. The apparatus of claim 29, wherein at least one of the means for storing, the means for updating, or the means for generating is integrated into at least one of an internet-of-things (IoT) device, an IoT device controller, or a portable electronic device.
